(19) United States
(12) Patent Application Publication
(10) Pub. No.: US 2012/0046607 A1
(43) Pub. Date: Feb. 23, 2012

(54) PLUG TO BE INTRODUCED INTO A PASSAGE SUCH AS A NASAL PASSAGE OR EAR CANAL

(76) Inventor: Svante Syk, Skovde (SE)

(21) Appl. No.: 13/145,155

(22) PCT Filed: Jan. 22, 2010
(86) PCT No.: PCT/SE2010/000013

§ 371 Date: Sep. 19, 2011

(30) Foreign Application Priority Data
Jan. 22, 2009 (SE) SE0900066-2

(51) Int. Cl. A61M 31/00

(52) U.S. Cl. 604/103.02

(57) ABSTRACT

Formable absorbent that is shaped into a plug for insertion into a passage, such as a nasal passage or an ear canal. This plug comprises a material that can be compressed, it thereafter expanding against a surrounding wall and generating a pressure against said wall, said plug having at least one through hole for the passage of air, said plug also being able to contain medicinal preparations for various purposes (e.g. stanching).

### TECHNICAL BACKGROUND

Some of the most usual forms of taking medicines are tablets, injections, plasters/patches and inhalers. This invention has proved extremely suitable for transferring substances to blood vessels by direct contact with a unit (plug) that secretes the substances in question.

In addition to the use as a plug in the nose to stanch nose bleeds, the invention set out in the present documentation opens new possibilities in medication. For example, the plug can be designed to impart active substances to the surface blood vessels in the nose.

During development of the plug to stanch nose bleeds (the original idea), said invention's/product's possible uses have increased. One of the new fields of application is that the plug can also be advantageously used in ear canals. This applies particularly to the administering of medication in ear canals.

It is common for children to have narrow ear canals. Furthermore, ear inflammations are common amongst babies. The present invention makes it possible to easily get into the ear to administer medication using, for example, a local anaesthetic. The pain can thus be reduced and the ease with which a medicinally prepared unit can be introduced into the ear canal is outstanding.

Furthermore, a nasal plug can also be designed to secrete or impart substances that can be borne by inhaled air and used to medicate the pharynx or lungs.

It is well known that problems arise with nose bleeds. Practically all adults have had nose bleeds and found them to be troublesome. There is a large group of people who regularly have light nose bleeds. The possible reasons for the bleeding include blood vessels that are close to the surface, allergies that irritate the nasal passages and great exertion or blows during sporting events, etc.

A smaller group of people suffers from heavy bleeding. Owing to large blood losses, the vomiting that occurs when blood runs down into the pharynx and fainting due to decreased blood pressure, such bleeding is extremely serious.

The most common procedure used when there is a nose bleed during, for example, a sporting event is to reduce the flow by pushing a cotton-tipped swab into the nose. There are several considerable disadvantages with this:
The nasal passage is obstructed and renders normal breathing (which is vital during exertion in sports events, etc.) impossible.
The blood coagulates in the cotton swab and new bleeding often occurs when the swab is removed.
A cotton swab can act as an irritant and is a considerable aesthetic disfigurement.
As they have no added agents, ordinary cotton swabs lack an efficient blood-stanching function.

There are several patents in the field of nose bleeds. This patent application is a further development of an earlier application by the same inventor. It is widely known that an idea constantly develops as findings amass during development into a commercial product. The consequence is often that the newly gained findings lead to a new patent application to protect the additions to the earlier invention. The previous application was SE0602534-0.

One example from other inventors is Marshall U.S. Pat. No. 5,383,891. In this, one or more oval units are pushed into the nasal passage. The units can be removed using a thread. A large “sticking plaster” is also used to cover the nose. The inserted oval units are sealed and do not allow any breathing. Using several accessories, the invention is cumbersome. The user is also highly conspicuous and, most likely, presents a frightening sight for children and many others.

Wilnelsia U.S. Pat. No. 6,666,211 comprises a “sticking plaster mask” that, shaped like a part of the nose, is used to stanch nose bleeds. No other areas of application are claimed for this. As with the previous example, wearing this “mask” makes the user highly conspicuous and the presented sight may be perceived as frightening. Furthermore, a risk is introduced for people who are vulnerable to contact allergies. On top of all this, it is doubtful whether the cited inventions can be used by people who, due to exertion (in sports events, etc.), are sweating. It is similarly doubtful that women and children appreciate the “disfigurement” involved in using the cited inventions.

The present invention solves several of the cited problems by allowing breathing through a hole (a tube or similar in the plug) and by it being possible to treat the plug with a coagulant. The user does not need the large “masks” and sticking plasters of the above-cited patents. Producing moulding tools for plasters and masks adds to manufacturing costs and, consequently, the resultant product is relatively expensive.

Yet a further major disadvantage of the above-cited patents is that they contain “facial-skin adhering” glues. Said glues are expensive and have a limited storage life.

During its development, the present invention has proved that it can also be used for administering medicines.

A further application is that people who snore as a result of narrow nasal passages can quite simply have a nasal passage expanded by using an expanding material in the plug. The plug also provides an increased breathing function where there are exceptional oxygenation demands, e.g. in extreme sports and/or illnesses.

### BRIEF ACCOUNT OF THE INVENTION

An absorbent comprising a square (or other shape) of a compress-like material is formed (by rolling) into a cylinder. The construction of the absorbent may comprise a somewhat harder core with an absorbent surface layer that, to reduce the risk of sticking to the surface of the wound or to nasal secretions, may be calendered. Owing to this construction, there is an in-built predisposition to “straightening out”. This function is used when the rolled-up absorbent is introduced into a nasal passage/ear canal. Here, it seeks to unroll and thus exerts pressure against the wall, which brakes the unrolling. This is favourable for counteracting nose bleeds (if this is the application in question). Furthermore, any medicinal substance in the outer layer can be pressed out by the unrolling and resultant pressure. If the application is to widen nasal passages in cases of snoring, this predisposition to unrolling is advantageous as it achieves the desired widening.

FIG. 4 shows an absorbent (41) in its original shape. FIG. 5 shows an absorbent (41) that, to facilitate rolling, has an integrated rod (51). FIG. 6 shows how rolling is preferably effected by hand and FIG. 7 shows how tweezers (71) or equivalent can be used. FIG. 8 shows a cylindrical rod (81) that has a slot (82). The absorbent (41) can be threaded into the slot and then rolled onto the rod for easy introduction into a nasal passage/ear canal. The absorbent can advantageously be equipped with a rod for easy location on insertion or removal.

Another design is to manufacture a plug from an equivalent material. A plug, possibly designed in several sizes to fit various noses and ages, is inserted into the nose during a nose bleed or when there is a need for medication. The plug comprises a body with a part that lets through air (e.g. a tube or similar equivalent hole), this body being surrounded by an absorbent mass that has a surface coating which prevents the plug getting stuck to surfaces. This is especially important where there are wounds, e.g. in a nose bleed. The plug's absorbent (of the super-absorbent type—fluff pulp or equivalent) expands in the nasal passage and thus exerts pressure on said passage where there are surface blood vessels. The tube or its equivalent allows breathing through said nasal passage.

The invention can be used in several ways. Some examples of these are described briefly below.

### 1. Nose Bleeds

The plug expanding against the nasal passage actively helps to stanch the blood flow. The incoming respiratory air that passes through the plug has, through its passing, a positive cooling effect and results in the blood vessel(s) contracting and, consequently, reducing the bleeding. A substance such as cyklokapron stanches bleeding, disinfects the surface of the wound and reduces the risk of the plug becoming stuck to said surface. As a preventive measure where there is a risk of bleeding, the nasal plug can also be inserted in the nose before bleeding occurs.

### 2. Secretion of Substances to Blood Vessels

The blood vessels in nasal passages lie close to the surface and are particularly suitable for taking up substances that may be on the surface of the plug or may be secreted to its surface. Insulin administration and an alternative to nicotine patches are two example uses.

### 3. Secretion of Substances to Respiratory Air

Because respiratory air passes through the plug, substances can also be integrated into, and released from, the plug with the purpose that said substances will be borne by inhaled air into the airways and eventually carried to the lungs. An example use here is allergy medicines that are often administered by spray or inhaler.

### 4. Secretion of Substances

Another possibility is for the plug to contain aromatics (aroma releasing substances) that counteract smells that may be troublesome in certain environments.

### 5. Filter Function

A design that filters air presents possibilities, e.g. filtration of pollen and similar small particles for people with allergies.

### 6. Anti-Snoring Device

Narrow, flaccid nasal passages can be a cause of snoring. This can be remedied by a plug with properties that, when the plug is used, result in the expansion of nasal passages.

### 7. Administering of Medication in Ear Canals

It is a known fact that babies often sustain inflammations in ear canals and suffer from these and the attendant pains. This invention can be used to administer analgesics in ear canals.

### SHORT DESCRIPTION OF THE FIGURES

The present invention is described in some detail with the assistance of the following figures:

FIG. 1 shows two possible designs.

FIG. 2 shows a normal and a compressed plug.

FIG. 3 shows the plug inserted in the nose.

FIG. 4 shows an absorbent as a flat sheet.

FIG. 5 shows an absorbent with a rod.

FIG. 6 shows a commenced rolling.

FIG. 7 shows an absorbent and tweezers.

FIG. 8 shows a tool for rolling.

### A FIRST DESIGN POSSIBILITY

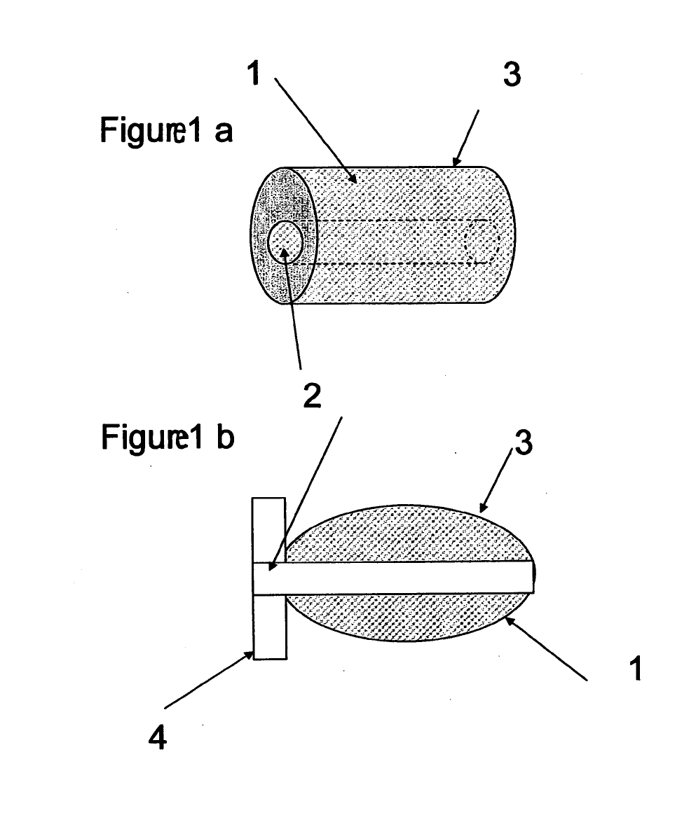

FIG. 1a shows a design where a cylindrical plug (1) has a tube (2) for air flows. Said tube (2) enables breathing. The plug (1) is made of a blood absorbent material and contains a coagulant, which is also on the surface (3). Alternatively, the plug can contain medicinal preparations that are intended to be taken up by the surface blood vessels against which the plug presses. The plug can also be designed to give off substances that are borne by inhaled air and carried to, for example, other airways, the pharynx or lungs.

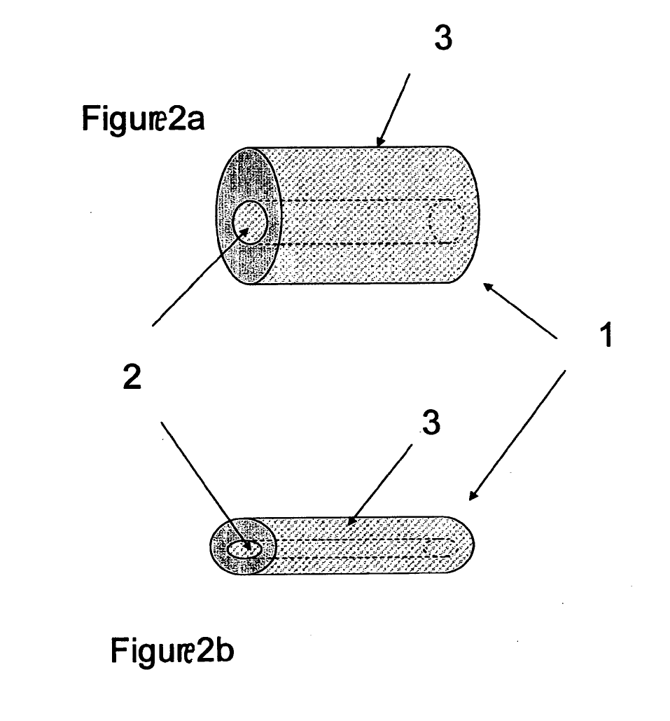

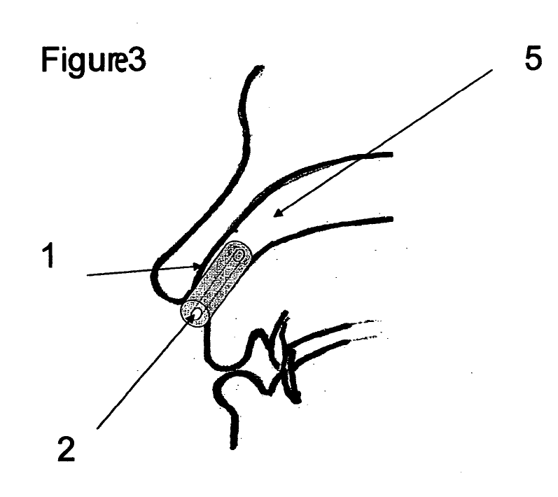

FIG. 1b shows an alternative design in which the tube (2) protrudes from the plug and has a collar (4). This collar (4) facilitates correct insertion of the plug (1) and simplifies removal. FIG. 2 shows a plug (1) that has been compressed for easier insertion in the nasal passage (5 in FIG. 3).

FIG. 3 shows how the plug sits in the nasal passage (5). FIG. 4 shows a flat absorbent (41) that is used for rolling up into a cylinder or plug.

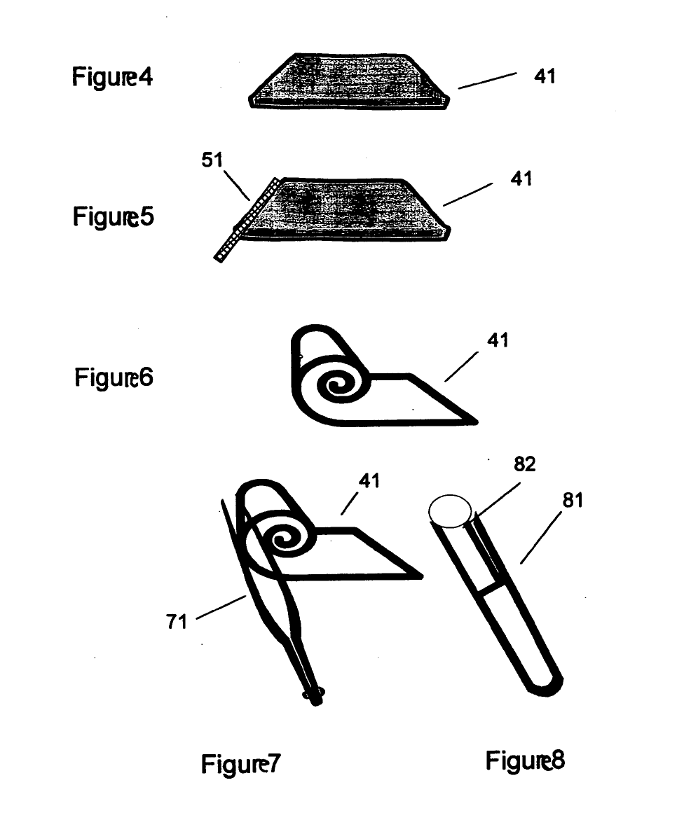

FIG. 5 shows how an absorbent (41) can have a rod (51), a “rolling rod”, integrated into the absorbent. FIG. 6 shows how rolling of the absorbent (41) has is been commenced. FIG. 7 shows a pair of tweezers (71) that can be used to roll the absorbent (41) up for subsequent insertion into a nasal passage/ear canal. FIG. 8 shows a tool comprising a rod (81) with a slot (82). One end of the absorbent can be threaded into said slot and the absorbent (41) then, for easy insertion into the nasal passage/ear canal, rolled around the rod (81).

The present description relates solely to one possible design. However, as various needs can influence the plug's design and construction, the designs and properties are not limited to those described above but extend to all those within the scope of this document.

1. Formable absorbent that is shaped as, or can be shaped into, a plug for insertion into a passage such as a nasal passage or an ear canal, the whole being characterised by: said plug comprising a material that can be rolled, or compressed, and which may thereafter expand against a surrounding wall (such as in a nasal passage or in an ear canal) thereby generating a pressure against said wall, said plug being able to have a through hole for the passage of air, said plug also being able to contain medicinal preparations for various purposes, the preparations being given off via air or via being taken up by surface blood vessels.

2. Formable absorbent as per patent claim 1, characterised by: said plug having properties that lead to its material filling out the shape of the passage/canal and pressing against the tissue of the passage/canal, it being possible for said plug to be comprised of a rolled material that, after insertion, unrolls to form a sleeve that has air ducts.

3. Formable absorbent as per patent claim 1, characterised by: said plug being treated with, and/or containing, preparations, medicines or similar that give off substances to blood vessels in quantities/doses that are measured to achieve certain desired effects.

4. Formable absorbent as per claim 1, characterised by: said plug being treated with, and/or containing, preparations, medicines or similar that are to be given off to the respiratory air and borne with said air to the pharynx, airways and, possibly, lungs.

5. Formable absorbent as per claim 1, characterised by: the above-mentioned hole being enclosed by a sealed surface that is formed when the cellular material is cast into a shape.

6. Formable absorbent as per claim 1, characterised by: said plug having material properties that lead to expansion in the nasal passage, thereby ensuring an opening and preventing flaccid tissue from flapping and generating a snoring noise.

7. Formable absorbent as per claim 1, characterised by: said plug—when made of a rolled material that, after insertion, unrolls to form a sleeve that has air ducts—being integrated with a rod for the rolling up and insertion into the passage/canal, said rod also being used on removing the plug from the passage/canal.

8. Formable absorbent as per claim 1, characterised by: the outward end being arranged, in its simplest form, to present a collar that provides a simple check against being sucked into the pharynx and which also provides an easy grip when removing.

9. Formable absorbent as per claim 1, characterised by: the constituent parts not subjecting the environment to any load and the parts being easy to destroy.
